(19) United States
(12) Patent Application Publication
(10) Pub. No.: US 2003/0124175 A1
(43) Pub. Date: Jul. 3, 2003

(54) Cicatrizant hydrocolloidal patch containing hyaluronic acid and chondroitin sulphate

(76) Inventors: Alberto Garavani, Massagno (CH); Irina Rapaport, Rovio (CH)

Correspondence Address: ABELMAN FRAYNE & SCHWAB, Attorneys at Law, 150 East 42nd Street, New York, NY, 10017, US

(21) Appl. No.: 10/104,410

(22) Filed: Mar. 21, 2002

(30) Foreign Application Priority Data
Mar. 22, 2001 (IT) MI2001A000611

(51) Int. Cl. A61K 9/70

(52) U.S. Cl. 424/449

(57) ABSTRACT

Cicatrizant hydrocolloidal patch comprising:

a) a support layer,

b) an adhesive layer containing an adhesive polymer, at least one hydrocolloid, hyaluronic acid or a pharmaceutical salt thereof, chondroitin sulphate or a pharmaceutical salt thereof,

c) a protective layer removable at the moment of use.

### FIELD OF THE INVENTION

The present invention concerns a cicatrizant hydrocolloidal patch and the relative preparation process.

### STATE OF THE ART

Cicatrizant pharmaceutical formulations for topical use, based on hyaluronic acid or a pharmaceutical salt thereof, have been known for some time.

For instance EP 0480198 describes pharmaceutical compositions containing the sodium salt of hyaluronic acid and antiseptic substances for topical use. However, these compositions are in hydrogel form and have the disadvantage of adding liquid to the wound when applied to it, hence making it even more difficult to eliminate the exudate from the wound.

These drawbacks are solved with the self-supporting dry transparent film described in international patent application WO 97/02845, consisting of a mixture of at least one hydrocolloid and hyaluronic acid.

This film is prepared with a process that envisages

preparation of a very diluted aqueous composition containing hyaluronic acid at concentrations between 0.5 and 2% in weight, and hydrocolloids at concentrations between 1% and 20% optionally in the presence of an additional solvent such as glycerol.

casting of the composition on a support,

exsiccation of all the liquid components of the aforesaid composition by treatment in a stove.

An anhydrous film is thus obtained in which the between 2 and 98% and the remaining components to 100% are the hydrocolloids. The complete elimination of the water, achieved by exsiccation in a stove requires extremely long heating times (in the order of several hours), with a consequent notable waste of energy. In addition, since with this type of procedure it is difficult to obtain a film of uniform thickness with a constant level of anhydricity, the industrial implementation of such a process is virtually impracticable.

Moreover, all the hydrocolloids, with the exclusion of polyvinylpyrrolidone, tend to worsen the mechanical properties of the self-supporting film, namely its tensile strength and elongation at break. To obviate such a drawback large quantities of the expensive hyaluronic acid must be added, in most cases, more than 10% and, in some cases, in the event that sodium alginate is used as the hydrocolloid, in amounts decidedly greater than 25%. Only when polyvinylpyrrolidone is employed it is possible to obtain good mechanical properties using smaller amounts of hyaluronic acid, which in any case must be around 2.5%.

In Italian patent 1301470 a cicatrizant hydrocolloidal patch is described comprising a support layer, an intermediate layer containing an adhesive polymer, at least one hydrocolloid and hyaluronic acid or a pharmaceutical salt thereof and, finally, a protective layer.

This patch does not show sufficient cicatrizant strength even at concentrations of hyaluronic acid in the order of 2% in weight out of the weight of the adhesive layer. In fact, the cicatrizant effect does not diverge, in a statistically significant manner, from the cicatrizant activity shown by the placebo patch, not containing any active principle.

In “Effect of chondroitin sulfate preparation on wound healing and strength of the surgical scar” by M. Fialkova et al BYULLETTIN EKSPERIMENTAL' NOY BIOLOGII I MEDITSINY, Vol. 108, N^{o}9 pp. 350-351 the results of an experiment carried out on a model of “full thickness” (300 mm²) cutaneous wound in rats are discussed, wherein such a wound has been treated with one or two applications of 30 mg of sodium chondroitin sulphate. The reduction of the damaged surface (measuring through planimetry) was more rapid in the treated group. For instance, 8 days after the second application the residual area in the treated group was half that of the control group, and also the clinical signs connected with the lesion (edema-exudate) decreased more quickly in the treated group.

The therapeutic form considered in this article diverges from that considered in the Italian patent since the active principle is administered intramuscularly by means of an injectable solution and at high concentrations of 10%.

### SUMMARY OF THE INVENTION

The Applicant has now unexpectedly discovered a cicatrizant hydrocolloidal patch containing hyaluronic acid and chondroitin sulphate as the active principle which, even at low concentrations of both chondroitin sulphate and hyaluronic acid, when it is applied to a wound allows to attain a cicatrization speed, expressed as percentage of reduction of the wound surface in time, comparable to that of bandages available on the market for the same purposes, namely CONVATEC® or VARIHESIVE-E®), but, unlike the latter, also promotes the formation of dermis and collagen production.

The object of the present invention is therefore a cicatrizant hydrocolloidal patch comprising:

a) a support layer,

b) an adhesive layer containing an adhesive polymer, at least one hydrocolloid, hyaluronic acid or a phrmaceutical salt thereof and chondroitin sulphate or a pharmaceutical salt thereof,

c) a protective layer removable at the moment of use.

### DETAILED DESCRIPTION OF THE INVENTION

The patch object of the present invention preferably contains hyaluronic acid in the form of one of its pharmaceutically acceptable salts at concentrations preferably between 0.01 and 5%, in weight out of the total weight of adhesive layer (b).

The molecular weight of the hyaluronic acid is preferably between 50,000 and 1,000,000.

The chondroitin sulphate in the patch of the present invention is preferably the bisodium salt of chondroitin-4-sulphate or chondroitin sulphate A, and the concentration of said active principle is preferably between 0.01% in weight and 5%. For simplification the aforesaid salt will hereafter be defined by the term sodium chondroitin sulphate.

According to a particularly preferred solution, the concentration of sodium hyaluronate in the patch according to the present invention is between 0.05 and 1%, and that of sodium chondroitin sulphate is between 0.05 and 1%.

In fact, it has surprisingly been found that, when such active principles have concentrations that fall within the aforesaid preferred intervals and particularly when sodium hyaluronate has a concentration of 0.2% and sodium chondroitin sulphate has a concentration of 0.3% in weight out of the total weight of adhesive layer (b), the patch of the invention shows a greater cicatrizant effect compared to that a patch of similar formulation, but containing sodium hyaluronate at a concentration of 2% and sodium chondroitin sulphate at a concentration of 3% in weight out of the total weight of the adhesive layer, and comparable to that of bandages available on the market such as VARIHESIVE®.

Preferred hydrocolloids for use in adhesive layer (b) of the patch according to the present invention are sodium carboxymethylcellulose of molecular weight of between 700 and 50,000, pectin USPL optionally mixed with saccharose, or mixtures thereof.

The concentration of said hydrocolloid is preferably between 10 and 90% in weight out of the total weight of adhesive layer (b).

According to a particularly preferred solution, a mixture of the following is used as a hydrocolloid: sodium carboxymethylcellulose, commercially available under the trade name of Blancosa® 7H4XF, sodium carboxymethylcellulose commercially available under the trade name of CEKOL®, Pectin USPL available under the trade name of GENU-PECTIN, added with saccharose (Sugar mix). This mixture of hydrocolloids is preferably present in adhesive layer (b) at concentrations of between 10 and 80%, even more preferably at concentrations of 47% in weight out of the total weight of said adhesive layer (b).

The adhesive polymer of layer (b) of the patch object of the present invention is preferably chosen between polyisobutylene of molecular weight of between 500 and 100,000, isoprene/styrene copolymer or mixtures thereof, at concentrations of between 10 and 90% in weight out of the total weight of adhesive layer (b).

According to a particularly preferred solution a mixture of polyisobutylene having a mean molecular weight of 40,000 and commercially available under the trade name of Oppanol® B15, and of styrene/isoprene copolymer Kraton® D-1107CS is used. The concentration of said adhesive polymeric mixture in layer (b) is preferably between 10 and 80%, even more preferably of 45% in weight out of the total weight of the adhesive layer (b).

The patch according to the present invention preferably contains a plasticizer chosen in the group consisting of mineral oil optionally with traces of white naphthenic oil, commercially available under the trade name of ENERPAR® and a mixture of polyterpenic resin and petroleum hydrocarbon resin, commercially available under the trade name of WINGTAC®10, and relative mixtures of said plasticizers at concentrations of between 0.5 and 25% in weight calculated out of the total weight of said adhesive layer (b). According to a preferred solution a mixture of the aforesaid mineral oil and of the mixture of polyterpenic resin/petroleum hydrocarbon resin is used and the total concentration of said plasticizer is between 1 and 10%, and even more preferably of 8% in weight out of the total weight of the adhesive layer (b).

In the patch according to the present invention preferably the support or layer (a) is made up of polyurethane as a film or a foam, while layer (c), which is the sheet removable at the moment of use, is preferably made of silicon paper.

The patch object of the present invention is preferably produced with a process that comprises the following steps:

i) dry mixing of hyaluronic acid or a pharmaceutical salt thereof and chondroitin sulphate or a pharmaceutical salt thereof with the hydrocolloid,

ii) mixing of the powders of the previous stage with the adhesive composition and optionally a plasticizer;

iii) extrusion of the paste deriving from step (ii) at a temperature of between 40 and 90° C., preferably of 80° C., between the support layer (a) and the removable protective layer (c).

Shown below are two illustrative but non-limiting examples of composition of the hydrocolloidal patch according to the present invention.

### EXAMPLE 1

Hydrocolloidal Patch Composed of:

1. layer (c): silicon paper=0.82 g/total weight of the patch

2. layer (b): adhesive=10.25g/total weight of the patch,

3. layer (a): polyurethane support film=0.62 g/total weight of the patch.

Composition of adhesive layer (b)

| | | % in weight out of |
|---|---|---|
| | | the total weight of |
| Trade name | Usual name | layer (b) |
| OPPANOL ® B15 | Polyisobutylene | 29.24 |
| KRATON ® D-1107CS | Styrene-isoprene | 15.59 |
| | copolymer | |
| BLANCOSA ® 7H4XF | Sodium | 17.55 |
| | carboxymethyl- | |
| | cellulose | |
| GENU-PECTIN | Pectin USPL | 11.70 |
| CEKOL ® 4000 | Sodium | 15.59 |
| | carboxymethyl- | |
| | cellulose | |
| SUGARMIX ® | Saccharose | 1.95 |
| WINGTAC ® 10 | Synthetic polyterpenic | 3.90 |
| | resin/petroleum | |
| | hydrocarbon resin | |
| ENERPAR ® | Mineral oil with traces | 3.90 |
| | of white naphthenic oil | |
| Sodium hyaluronate | | 0.23 |
| Sodium chondroitin | | 0.35 |
| sulphate | | |

### EXAMPLE 2

| | | % in weight out of |
|---|---|---|
| | | the total weight of |
| Trade name | Usual name | layer (b) |
| OPPANOL ® B15 | Polyisobutylene | 27.78 |
| KRATON ® D-1107CS | Styrene-isoprene | 14.82 |
| | copolymer | |
| BLANCOSA ® 7H4XF | Sodium | 16.67 |
| | carboxymethylcellulose | |
| GENU-PECTIN | Pectin USPL | 11.11 |
| CEKOL ® 4000 | Sodium | 14.82 |
| | carboxymethylcellulose | |
| SUGARMIX ® | Saccharose | 1.85 |
| WINGTAC ® 10 | Synthetic polyterpenic | 3.70 |
| | resin/petroleum | |
| | hydrocarbon resin | |
| ENERPAR ® | Mineral oil with traces | 3.70 |
| | of white naphthenic oil | |
| Sodium hyaluronate | | 2.22 |
| Sodium chondroitin | | 3.33 |
| sulphate | | |

After cutting, each patch was sealed in a special airtight blister pack and irradiated with γ rays (normally between 25 and 50 KGy)

1—Macroscopic Appearance of the Wounds and Morphometric Analysis

Methodology

Dunkin Hartley type guinea pigs were used for this test.

A rectangular wound of 12 cm² (4×3) was made on one side of each guinea pig (10 guinea pigs+1 extra per group), maintaining the panniculus carnosus.

The medication was applied to the wound each day up to day 33 (end of the experiment).

The action of the medication and the appearance of the wounds were macroscopically analyzed following a scale of criteria based on moisture, adherence to the wound, inflammatory and haemorrhagic process and level of cicatrization.

A photograph was taken every two or three removals of the medication under standard conditions in order to automatically highlight the progress of the surface of the wound with an image analyser.

The following types of medication were tested.

group A: placebo hydrocolloidal patch

group B: hydrocolloidal plasters of example 1

group C: hydrocolloidal plaster of example 2

group D: VARIHESIVE® patch

Results

a) Macroscopic Appearance of the Wounds

The wounds of groups A, B, and C were moist, dark and sanguinolent for most of the time, whereas the wounds of group D were moist but not as dark as those in the other groups and also less sanguinolent. However, in the latter case some yellow liquid was observed in the wounds after day 5.

A certain tendency towards better cicatrization was observed in group B when compared to group A. In fact, the wounds in group B were smaller and had a better macroscopic aspect, since covered by a thinner scab.

b—Morphometric Analysis of the Surface of the Wounds

b-1 Surface of the Skin of the Different Groups

TABLE I
evolution of the mean surface of the wound in different groups (cm²)

| Day | GROUP A | GROUP B | GROUP C | GROUP D |
|---|---|---|---|---|
| 1 | 13.60 | 13.89 | 13.41 | 12.83 |
| 5 | 10.26 | 9.58 | 9.59 | 8.87 |
| 7 | 9.88 | 8.27 | 9.34 | 6.76 |
| 11 | 6.73 | 5.45 | 5.41 | 4.92 |
| 15 | 5.01 | 3.99 | 4.57 | 3.74 |
| 19 | 4.27 | 3.21 | 3.82 | 3.10 |
| 24 | 3.17 | 2.54 | 3.15 | 2.37 |
| 28 | 2.87 | 2.30 | 2.82 | 2.32 |
| 31 | 2.54 | 1.83 | 2.49 | 2.33 |
| 33 | 2.48 | 1.72 | 2.38 | 1.72 |

The results reported above show that in all groups the surface of the wound was reduced of approximately 50% in the first 11 days and was reduced more slowly afterwards.

A difference in the speed of cicatrization is observed between the different groups:

50% cicatrization of the surface for groups B and D at 8.3 and 8 days respectively, while in groups A and C this cicatrization is obtained at 10.9 and 9.4 days respectively.

75% cicatrization of the surface of the skin for groups B and D at 18.6 and 18.9 days respectively, while for groups A and C this value is reached at 23.8 and 23.7 days respectively.

The speed in cicatrization is better in groups B and D.

b-2—Statistical Analysis

Statistical analysis of the residual surface (non-parametric Mann & Whitney test) did not show any significant difference (p<0.05) between groups B and D, while cicatrization in groups A and C showed the same course.

2—Histological Examination

Methodology

The experiment was stopped at day 33 and the skin of three animals was taken for each group. After fixing with 10% formaldehyde, the samples were then denatured in alcohol solutions of increasing concentration and afterwards incorporated in paraffin.

Two pairs in a series, of approx. 5 μm thickness, were made on each sample with the aid of a HM350 microtome. The sections were dyed according to a modified trichromium technique for classic histopathological analysis and with toluidine blue to highlight metachromatic oxydic structures.

The pairs of histological samples were observed using a Polyvar microscope (Reichert) fitted with a 4, 10 and 25 objective with the possibility of adding a 1.25 lens.

Results

The wounds of the animals of group A and group D give similar results from a histological point of view.

Hypervascularization and exudates consisting of red blood cells and the presence of a foamy collagen-based matrix in the deep layers of the granulation tissue, were found in both groups. However, group A was associated with a more marked hypervascularization and inflammatory component when compared to group D.

The wounds of groups B and C show similar characteristics with respect to those of groups A and D, but, in addition, in the former ones the presence of a more mature deep dermis is observed. The collagen is denser and very similar to the adjacent layer of the normal dermis. This is particularly marked for the wounds of group C.

However, this group also shows a relative superficial granulation tissue of inflammatory type with a weakening of the epidermization process when compared to the wounds of group B.

Therefore, the active principles of the patches applied to groups B and C show a twoford effect, that is:

1) improved synthesis of collagen and organization in the deep layers of the wounds of groups (B) and in particular (C),

2) a more marked superficial inflammatory component associated with these active principles in the case of the groups (C).

1. Cicatrizant hydrocolloidal patch comprising
a) a support layer,
b) an adhesive layer containing an adhesive polymer, at least one hydrocolloid, hyaluronic acid or a pharmaceutical salt thereof, chondroitin sulphate or a pharmaceutical salt thereof,
c) a protective layer removable at the moment of use.

2. The patch as in claim 1, containing hyaluronic acid in the form of one of its pharmaceutically acceptable salts at concentrations of between 0.01 and 5% in weight out of the total weight of adhesive layer (b).

3. The patch as in claim 2, containing the sodium salt of hyaluronic acid at concentrations of between 0.05% and 1% in weight out of the total weight of adhesive layer (b).

4. The patch as in claim 1, wherein the molecular weight of hyaluronic acid is preferably between 50,000 and 1,000,000.

5. The patch as in claim 1, wherein the chondroitin sulphate in the patch is sodium chondroitin sulphate, and the concentration of said active principle is between 0.01% in weight and 5% in weight out of the total weight of adhesive layer (b).

6. The patch as in claim 1, containing sodium hyaluronate at concentrations between 0.05 and 1% and sodium chondroitin sulphate at concentration between 0.05 and 1% in weight out of the total weight of adhesive layer (b).

7. The plaster as in claim 6, wherein the concentration of sodium hyaluronate is 0.2% and that of sodium chondroitin sulphate is 0.3% out of the total weight of adhesive layer (b).

8. The patch as in claim 1, wherein the hydrocolloid is chosen from the group consisting of sodium carboxymethylcellulose of molecular weight of between 700 and 50,000, pectin USPL optionally mixed with saccharose, or relative mixtures thereof.

9. The patch as in claim 8, wherein the concentration of said hydrocolloid is between 10 and 90% in weight out of the total weight of adhesive layer (b).

10. The patch as in claim 1, wherein said hydrocolloid is a mixture of sodium carboxymethylcellulose and pectin USPL added with saccharose at concentrations of between 10 and 80% in weight out of the total weight of adhesive layer (b).

11. The patch as in claim 10, wherein the concentration of said hydrocolloid is between 70 and 90% in weight out of the total weight of adhesive layer (b).

12. The patch as in claim 10, in which the concentration of said hydrocolloid is equal to 47% in weight.

13. The patch as in claim 1, wherein the adhesive polymer of layer (b) is chosen between polyisobutylene of molecular weight of between 500 and 100,000, isoprene/styrene copolymer, or relative mixtures thereof, at concentrations of between 10 and 90% in weight out of the total weight of adhesive layer (b).

14. The patch as in claim 1, wherein the adhesive polymer of layer (b) consists of a mixture of polyisobutylene with a molecular weight of 40,000 and of styrene/isoprene copolymer at a concentration of between 10 and 80% in weight out of the total weight of adhesive layer (b).

15. The patch as in claim 14, wherein the concentration of said polymeric mixture is equal to 45% in weight out of the total weight of adhesive layer (b).

16. The patch as in claim 1, further containing a plasticizer chosen from the group consisting of mineral oil optionally with traces of white naphthenic oil, a mixture of petroleum hydrocarbon resin and polyterpenic resin, and relative mixtures of said plasticizers at concentrations of between 0.5 and 25% in weight calculated out of the total weight of said adhesive layer (b).

17. The patch as in claim 1, further containing a plasticizer containing a mixture of mineral oil optionally with races of white naphthenic oil with a mixture of polyterpenic resin/petroleum hydrocarbon resin, said plasticizer being at a concentration between 1 and 10% in weight out of the total weight of adhesive layer (b).

18. The patch as in claim 17, wherein the concentration of said plasticizer is approx. 8% in weight out of the total weight of adhesive layer (b).

19. The patch as in claim 1, wherein the support layer (a) consists of polyurethane as a film or foam.

20. The patch as in claim 1, wherein layer (c), the sheet removable at the moment of use, is preferably made of silicon paper.

21. Process to prepare the patch of claim 1 comprising the following steps:
i) dry mixing of the hyaluronic acid and chondroitin sulphate with the hydrocolloid,
ii) mixing of the powders of the previous stage with the adhesive composition and optionally a plasticizer;
iii) extrusion of the paste deriving from step (ii) at a temperature of between 40 and 90° C. between support layer (a) and the removable protective layer (c).

22. The process of claim 21, wherein the temperature of stage (iii) is 80° C.
